(19) United States
(12) Patent Application Publication
(10) Pub. No.: US 2022/0071755 A1
(43) Pub. Date: Mar. 10, 2022

(54) TRANSFER DEVICE

(71) Applicants: BOE Regenerative Medicine Technology Co., Ltd, Beijing (CN); BOE Technology Group Co., Ltd., Beijing (CN)

(72) Inventors: Yufei Zhao, Beijing (CN); Dehua Chang, Beijing (CN); Donghua Liu, Beijing (CN); Yang Liu, Beijing (CN); Shuai Liu, Beijing (CN)

(21) Appl. No.: 17/458,072

(22) Filed: Aug. 26, 2021

(30) Foreign Application Priority Data
Sep. 9, 2020 (CN) 202021960667.3

(51) Int. Cl. A61F 2/00; A01N 1/02

(52) U.S. Cl. CPC A61F 2/0095; A01N 1/0273

(57) ABSTRACT

A transfer device includes a transfer device body, the transfer device body includes a carrying plate and a picking-up plate. The picking-up plate is configured to drive the carrying plate to move, and a first end of the picking-up plate is fixedly connected to a first edge portion of the carrying plate. The carrying plate is configured to pick up and carry an object to be transplanted.

### CROSS-REFERENCE TO RELATED APPLICATION

This application claims priority to Chinese Patent Application No. 202021960667.3, filed on Sep. 9, 2020 and entitled “TRANSFER DEVICE,” the disclosure content of which is incorporated herein by reference in its entirety.

### TECHNICAL FIELD

The present disclosure relates to the field of operation technologies in clinical applications, clinical trials, clinical experiments and biological experiments, and in particular, relates to a transfer device.

### BACKGROUND

In operations of clinical applications, clinical trials, clinical experiments and biological experiments, it is often need to take out a thinner object to be transplanted (also called a sheet, which is not distinguished in this application), such as a lamellar tissue or a cell membrane layer, from a transport box, and transplant the object to be transplanted into an animal body, a human body or other vessels for subsequent operations. Therefore, how to transfer the object, to be transplanted is an urgent problem to be solved.

### SUMMARY

Embodiments of the present disclosure provide a transfer device, which includes following technical solutions.

In one aspect of the embodiments of the present disclosure, a transfer device is provided. The transfer device includes a transfer device body, wherein the transfer device body includes a carrying plate and a picking-up plate; wherein

the picking-up plate is configured to drive the carrying plate to move, and a first end of the picking-up plate is fixedly connected to a first edge portion of the carrying plate; and

the carrying plate is configured to pick up and carry an object to be transplanted.

In some embodiments, the carrying plate and the picking-up plate are connected by a rotating mechanism, wherein the rotating mechanism is configured to cause, in response to being rotated, an angle between the carrying plate and the picking-up plate to change.

In some embodiments, the transfer device body further includes a fixing member, wherein the fixing member is configured to fix the angle between the carrying plate and the picking-up plate to a changed angle in the case that the angle between the carrying plate and the picking-up plate is changed.

In some embodiments, the angle between the picking-up plate and the carrying plate is greater than 90° and less than 180°.

In some embodiments, the carrying plate and the picking-up plate are formed to an integral structure.

In some embodiments, the carrying plate has at least one liquid leaking hole, wherein the liquid leaking hole is configured to allow a liquid on the carrying plate to leak.

In some embodiments, the transfer device body further includes at least one blocking plate, wherein the blocking plate is configured to block the object to be transplanted from sliding out of the carrying plate, and an end of the blocking plate is fixedly connected to a second edge portion of the carrying plate.

In some embodiments, the first edge portion is not overlapped with the second edge portion.

In some embodiments, the blocking plate is provided with at least one liquid leaking hole, wherein the liquid leaking hole is configured to allow a liquid on the carrying plate to leak.

In some embodiments, the liquid leaking hole is at least one of a polygonal hole and a circular hole.

In some embodiments, the carrying plate, the picking-up plate, and the blocking plate are formed to an integral structure.

In some embodiments, the transfer device further includes an auxiliary baffle, wherein the auxiliary baffle is configured to assist transfer of the object to be transplanted to the carrying plate.

In some embodiments, a shape of a first end of the auxiliary baffle is matched with a shape of a third edge portion of the carrying plate.

In some embodiments, the carrying plate further includes a second edge portion configured to fix a blocking plate, and the third edge portion is not overlapped with the first edge portion and the second edge portion.

In some embodiments, the first edge portion, the second edge portion, and the third edge portion constitute an entire edge of the carrying plate.

In some embodiments, the transfer device further includes a buffer liquid conveying device, wherein the buffer liquid conveying device is configured to output a buffer liquid, such that the object to be transplanted moves under driving of the buffer liquid.

In some embodiments, the buffer liquid conveying device is fixed to the picking-up plate, and the buffer liquid delivering device is provided with a liquid outlet, wherein the liquid outlet is disposed at an end, proximal to the carrying plate, of the buffer liquid delivering device.

In some embodiments, the carrying plate is made of a non-transparent material.

In some embodiments, a carrying surface of the carrying plate is a circular surface.

In some embodiments, a manufacturing material of the transfer device includes any one of stainless steel, titanium alloy, virgin polystyrene, and high borosilicate glass.

### BRIEF DESCRIPTION OF THE DRAWINGS

FIG. 1 is a schematic structural diagram of a transfer device according to an embodiment of the present disclosure; and

FIG. 2 is a schematic structural diagram of another transfer device according to an embodiment of the present disclosure.

### DETAILED DESCRIPTION

In order to make the objectives, technical solutions, and principles of embodiments of the present disclosure more clearly, the technical solutions of embodiments of the present disclosure will be clearly and completely described below in conjunction with accompanying drawings of the embodiments of the present disclosure. It is apparent that the described embodiments are not all of but a part of the embodiments of the present disclosure. All the other embodiments achieved by those of ordinary skills in the art based on the embodiments of the present disclosure without creative work shall fall within the protection scope of the present disclosure.

Unless otherwise defined, technical terms or scientific terms used in the present disclosure shall be comprehended as the common meaning understood by those of ordinary skills in the art, The terms “first,” “second,” and the like used in the present disclosure do not denote any sequence, quantity, or importance, but are used to distinguish different parts or elements. The terms “include,” “comprise,” and the like mean that the elements or objects preceding the terms encompass the elements or objects and their equivalents as listed after these terms, and does not exclude other elements or objects. The terms “connected with,” “connected to,” and the like are not limited to physical or mechanical connections, but may include electrical connections regardless the connection is direct or indirect. The terms “upper,” “lower,” “left,” “right” and the like are only used to indicate the relative positional relationship, and when the absolute position of the object to be described is changed, the relative positional relationship may also change accordingly.

For clarity and conciseness of the following description of embodiments of the present disclosure, the present disclosure omits the detailed descriptions of known functions and known parts or elements.

The transfer device according to the embodiment of the present disclosure is suitable for transferring a sheet from a transport box to other vessels, transferring a sheet between different vessels, transplanting a sheet from a vessel to a surface of a visceral organ, or other similar scenarios. In addition, the sheet, before transferring or transplanting, is in a tissue fluid configured to protect the sheet.

At present, the way to transfer the sheet is to dump the transport box to thereby transfer the sheet from the transport box to the vessel. However, due to the tissue fluid in the transport box, the sheet in the transport box may be easily curled in the flowing tissue fluid during the dumping process, and may be subject to tears, wrinkles, and other unrecoverable or irreversible damages, which may cause a waste of resources. Moreover, in the process of clinical transplantation, it is also necessary to transplant the sheet to a specific position of the surface of the visceral organ. The transplanting process herein is also realized by dumping. However, the dumping may fail to accurately fix and flatten the sheet in a specific position, thereby resulting in a lower transplantation efficiency.

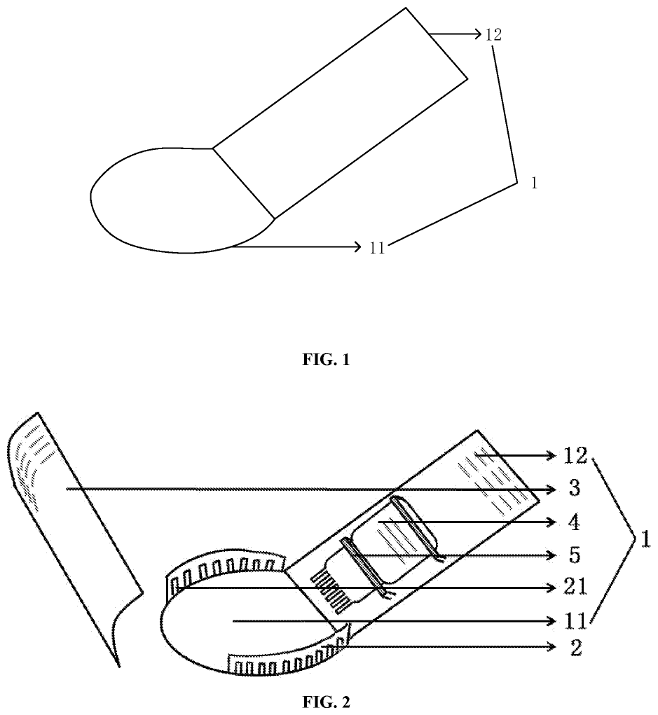

An embodiment of the present disclosure provides a transfer device. FIG. 1 is a schematic structural diagram of a transfer device. As shown in FIG. 1, the transfer device includes a transfer device body 1, wherein the transfer device body 1 includes a carrying plate 11 and a picking-up plate 12.

The carrying plate 11 is configured to move under driving of the picking-up plate to pick up the object to be transplanted and carry the picked-up object. In some embodiments, the carrying plate 11 may be disposed in a polygonal shape or a circular shape. In addition, a size of the carrying plate 11 should be larger than a size of the sheet to ensure that the sheet is completely disposed on the carrying plate 11 in a flattened state. But at the same time, the size of the carrying plate 11 should be smaller than a size of an opening of the transport box, such that the sheet may be picked up directly by the carrying plate 11 from the transport box. While designing the carrying plate 11, the size of the carrying plate 11 may be determined based on the size of the sheet in an expected application scenario of the carrying plate 11, and then the carrying plate 11 is manufactured based on the size as designed.

The picking-up plate 12 is mainly configured to drive the carrying plate 11 to move. A first end of the picking-up plate 12 is fixedly connected to a first edge portion of the carrying plate 11. Of course, the first end of the picking-up plate 12 and the first edge portion of the carrying plate 11 may also be connected by a rotating mechanism such as a buckle or a pivotable portion. The rotating mechanism herein is configured to cause, in response to being rotated, the angle between the carrying plate 11 and the picking-up plate 12 to change.

In addition, in the case that the carrying plate 11 and the picking-up plate 12 are connected by the rotating mechanism, the transfer device body 1 may further include a fixing member. The fixing member is configured to fix the angle between the carrying plate 11 and the picking-up plate 12 to a changed angle in the case that the angle between the carrying plate 11 and the picking-up plate 12 is changed. In some embodiments, in response to changing the angle between the carrying plate 11 and the picking-up plate 12 by rotating the rotating mechanism, the operator may further adjust the fixing member to keep the angle between the carrying plate 11 and the picking-up plate 12 at the changed angle via the fixing member, so as to prevent the angle between the carrying plate 11 and the picking-up plate 12 from changing during the transfer process. The operator may adjust the angle between the carrying plate 11 and the picking-up plate 12 based on the operator's own operating habit or the size of the transport box receiving the sheet. In some embodiments, the angle between the picking-up plate 12 and the carrying plate 11 may be greater than 90° and less than 180°.

In some embodiments, in order to facilitate the production, the carrying plate 11 and the picking-up plate 12 may be integrally formed. The carrying plate 11 and the picking-up plate 12 are formed to an integral structure at this point.

Furthermore, a second end of the picking-up plate 12 may be provided with a protrusion to facilitate the grasping for the operator. When the operator grasps the second end of the picking-up plate 12, the protrusion may generate relative resistance to the operator's hand, which thereby facilitates grasping of the transfer device body 1 for the operator and prevents the transfer device body 1 from slipping off the hand of the operator. The second end of the picking-up plate 12 is opposite to the first end of the picking-up plate 12.

Considering that the sheet, before the transferring or transplanting, is in the corresponding tissue fluid, the carrying plate 11 may simultaneously carry residual tissue fluid in the process of transferring the sheet by the transfer device according to the embodiment of the present disclosure, which may cause the sheet on the carrying plate 11 to flow with the tissue fluid. Therefore, as shown in FIG. 2, the transfer device body may further include at least one blocking plate 2 configured to block the object to be transplanted from sliding out of the carrying plate 11. In some embodiments, the blocking plate 2 is configured to prevent the sheet from flowing out of the carrying plate 11 along with the tissue fluid in the case that the tissue fluid flows out of the carrying plate 11. An end of the blocking plate 2 is fixedly connected to the second edge portion of the carrying plate 11. In some embodiments, the first edge portion is not overlapped with the second edge portion. In some embodiments, the carrying plate 11, the picking-up plate 12 and the blocking plate 2 may be formed to an integral structure.

The blocking plate 2 may be provided with at least one liquid leaking hole 21, wherein the liquid leaking hole 21 is configured to allow a liquid on the carrying plate 11 to leak, such that the residual tissue fluid on the carrying plate 11 is discharged via the liquid leaking hole 21 while carrying the sheet. In the case that the blocking plate 2 is disposed with the liquid leaking hole 21, the liquid leaking hole 21 may be formed in any shape. In some embodiments, the liquid leaking hole 21 may be at least one of a polygonal hole (such as a square hole) and a circular hole, as long as the tissue fluid on the carrying plate 11 can flow out. The number and size of the liquid leaking holes 21 disposed on the blocking plate 2 may be determined based on the size of the carrying plate 11, such that the tissue fluid remaining on the carrying plate 11 may not take too long to flow out of the carrying plate 11 via the liquid leaking hole 21.

Furthermore, in order to allow the tissue fluid on the carrying plate 11 to flow out as much as possible, the liquid leaking hole 21 may be disposed at a connection positon between the blocking plate 2 and the carrying plate 11. As shown in FIG. 2, the shape of the liquid leaking hole 21 is rectangle, and the liquid leaking hole 21 is disposed at the connection positon between the blocking plate 2 and the carrying plate 11. In this way, the tissue fluid remaining on the carrying plate 11 may be directly discharged via the liquid leaking hole 21 as much as possible, which thereby requires no operation from the operator such as tilting the carrying plate 11 to discharge the tissue fluid.

In addition, the blocking plate 2 may have a height to ensure that the sheet does not slide over the blocking plate 2 or fall off the carrying plate 11 in the case that the carrying plate 11 is tilted during the process of transferring the sheet.

Similarly, a liquid leaking hole may also be disposed on the carrying plate 11 to further ensure that the tissue fluid on the carrying plate 11 flows out completely. Furthermore, in the case that a liquid leaking hole is disposed on the carrying plate 11, the blocking plate 2 may optionally be disposed with or not disposed with a liquid leaking hole 21, which is not specifically limited in the embodiment of the present disclosure. In the case where a liquid leaking hole is disposed on the carrying plate 11 and no liquid leaking hole 21 is disposed on the blocking plate 2, the blocking plate 2 is configured to prevent the sheet from sliding out of the carrying plate 11.

Furthermore, the transfer device further includes an auxiliary baffle 3, the auxiliary baffle is configured to assist transfer of the object to be transplanted to the carrying plate 11. In some embodiments, in order to ensure the auxiliary effect of the auxiliary baffle 3, a shape of the first end of the auxiliary baffle 3 is matched with a shape of the third edge portion of the carrying plate 11. In some embodiments, the first end of the auxiliary baffle 3 and the third edge portion of the carrying plate 11 may both be arc-shaped and have a same arc length to ensure a seamless fit between the auxiliary baffle 3 and the carrying plate 11, thereby preventing the sheet from sliding off as caused by the gap formed due to the shape mismatch.

In some embodiments, the second end of the auxiliary baffle 3 distal from the carrying plate 11 may further include a protrusion. When the operator grasps the second end of the auxiliary baffle 3, the protrusion may generate relative resistance to the hand of the operator, which facilitates grasping of the auxiliary baffle 3 for the operator and prevents the auxiliary baffle 3 from slipping off the hand of the operator. The second end of the auxiliary baffle 3 herein is opposite to the first end of the auxiliary baffle 3.

The third edge portion of the carrying plate 11 may be not overlapped with the first edge portion of the carrying plate 11 and the second edge portion of the carrying plate 11, which thereby allows the auxiliary baffle 3 to effectively match with the carrying plate 11. In this embodiment, the first edge portion of the carrying plate 11, the second edge portion of the carrying plate 11, and the third edge portion of the carrying plate 11 may constitute the entire edge of the carrying plate 11. In some embodiments, in the case that one blocking plate 2 is provided and the auxiliary baffle 3 is matched with the carrying plate 11, edges of the edge portion connected to the auxiliary baffle 3, the edge portion connected to the picking-up plate 12, and the edge portion connected to the blocking plate 2 may be connected in sequence. In some embodiments, in the case that two blocking plates 2 are provided and the auxiliary baffle 3 is matched with the carrying plate 11, the two blocking plates 2 may both be disposed between the auxiliary baffle 3 and the carrying plate 11, which is namely the case shown in FIG. 2, and edges of the edge portion connected to the auxiliary baffle 3, the edge portion connected to the picking-up plate 12, and the edge portion connected to the blocking plate 2 may be connected to each other. In this way, under an effective matching of the carrying plate 11, the picking-up plate 12, the blocking plate 2 and the auxiliary baffle 3, the sheet may be prevented from sliding off between the auxiliary baffle 3 and the carrying plate 11, between the auxiliary baffle 3 and the blocking plate 2, between the blocking plate 2 and the carrying plate 11, between the auxiliary baffle 3 and the picking-up plate 12, or between the blocking plate 2 and the picking-up plate 12 during the transfer process.

In some embodiments, in order to facilitate the production, the carrying plate 11, the picking-up plate 12, and the blocking plate 2 may be integrally formed for manufacturing. An end, proximal to the carrying plate 11, of the picking-up plate 12 and an end, proximal to the carrying plate 11, of the auxiliary baffle 3 may further be disposed with a liquid leaking hole to facilitate the outflow of the tissue fluid on the carrying plate 11, which is not specifically limited in the embodiment of the present disclosure.

In addition, in the case that all the tissue fluid on the carrying plate 11 has flowed out, the sheet may fail to slide off the carrying plate 11 while transferring the sheet on the carrier plate 11 from the carrying plate 11 to the surface of other utensils or visceral organs due to the possible presence of a certain mutual adsorption force between the sheet and the carrying plate 11, which may cause the sheet cannot be transferred. Thus, as shown in FIG. 2, the transfer device according to the embodiment of the present disclosure may further include a buffer liquid conveying device 4, the buffer liquid conveying device is configured to output a buffer liquid, such that the object to be transplanted may move under driving of the buffer liquid and may for example flow out of the carrying plate 11.

In some embodiments, the buffer liquid conveying device 4 may be fixed to the picking-up plate 12 by a fixing device 5 disposed on the picking-up plate 12. Of course, the buffer liquid conveying device 4 may be detachable from the picking-up plate 12. The fixing device 5 may be implemented in a fashion of a fixing frame, a fixing strap, and the like. Alternatively, in the case that the buffer liquid conveying device 4 is a metal device, a magnet may be disposed on the picking-up plate 12 to realize fixing of the buffer liquid conveying device 4 by the adsorption force of the magnet. It should be noted that the buffer liquid conveying device 4 may not be fixed to the picking-up plate 12. In some embodiments, the buffer liquid conveying device 4 and the picking-up plate 12 may be independent structures. The buffer liquid conveying device 4 may be moved to a required position in the case that the buffer liquid conveying device 4 is required, and further outputs the buffer liquid, such that the object to be transplanted moves under the driving of the buffer liquid.

The buffer liquid delivering device 4 may be loaded with liquid or allow the liquid to pass through, and the buffer liquid conveying device 4 has a liquid outlet. In the case that the buffer liquid conveying device 4 is fixed to the picking-up plate 12, the liquid outlet of the buffer liquid conveying device 4 may be disposed at an end, proximal to the carrying plate, of the buffer liquid conveying device 4, such that the liquid in the buffer liquid conveying device 4 flows to the carrying plate 11 via the liquid outlet. Thus, the sheet tissue adsorbed on the surface of the carrying plate 11 may be washed by the flowing liquid, which prevents the sheet from being attached to the carrying plate 11 and enables the liquid to drive the sheet to flow out of the carrying plate 11. The liquid herein may be tissue fluid, or other liquids that do not damage sheet tissues, such as physiological saline.

It is worth noting that, as shown in FIG. 2, the buffer liquid conveying device 4 may be provided with a plurality of liquid outlets arranged side by side, such that the buffer liquid may evenly flow to the carrying plate 11 via the plurality of liquid outlets side by side, such that the sheet can be washed more uniformly.

In some embodiments, the buffer liquid conveying device 4 may be a buffer bottle. The buffer bottle may be made of a material with elastic recovery. That is, the buffer bottle may be squeezed in response to receiving an external force, and may restore the original shape in response to disappearance of the external force. During the use, the operator may squeeze the buffer bottle to reduce the air pressure in the buffer bottle, and then place the liquid outlet of the buffer bottle in a container carrying the liquid. Afterwards, the operator stops squeezing the buffer bottle, and at this point, the buffer bottle begins to restore the original shape. In the process of restoring the original shape of the buffer bottle, a certain air pressure difference may be generated at the liquid outlet of the buffer bottle, and the liquid in the container may enter the buffer bottle via the liquid outlet under the action of the air pressure difference. Then, the buffer bottle may be fixed to the picking-up plate 12, and during the process of controlling the sheet to slide off the carrying plate 11, the buffer bottle is pressed to allow the liquid in the buffer bottle to flow to the carrying plate 11. The buffer bottle herein may optionally contain 3-8 ml of liquid.

In some other embodiments, the buffer liquid conveying device 4 may be a communication device having a liquid inlet (such as, an uncovered box having a liquid outlet at the bottom). In the case that the buffer liquid delivering device 4 is fixed to the picking-up plate 12, the liquid inlet may be disposed at an end distal from the carrying plate 11, and in the case that the liquid is poured into the buffer liquid conveying device 4 via the liquid inlet, the liquid may flow to the carrying plate 11 via the liquid outlet.

Compared with the buffer liquid conveying device 4 implemented by the buffer bottle, the operator may control the amount of liquid flowing to the carrying plate 11 conveniently and flexibly in the case that the buffer liquid conveying device 4 is implemented by the communication device. Of course, the buffer liquid conveying device 4 may be implemented in other fashions, as long as there is a certain amount of liquid being transferred to the carrying plate 11 to assist the sheet on the carrying plate 11 to slide off.

During the use, in the case that a part of the sheet has slid off (i.e., a part of the sheet is on the carrying plate 11) and the other part has been separated from the carrying plate 11 and placed on the surface of the visceral organ to be transplanted, the sheet that has been separated from the carrying plate 11 may be pressed by an end of the auxiliary baffle 3 or a convex surface of the auxiliary baffle 3 to assist the sheet to be fixed at a specific position of the surface of the visceral organ to be transplanted. At this point, the transfer device body 1 may be moved in an opposite direction of the auxiliary baffle 3 to help the sheet to quickly separate from the carrying plate 11 to thereby complete the transplantation.

In some embodiments, a shape of the surface of the carrying plate 11 may be circle. In this way, the shape of the third edge portion of the carrying plate 11 may be circular arc, such that the sheet may be prevented from being scratched by the third edge portion in shapes having such as a corner during the process of sliding the sheet from the carrying plate 11 to the surface of other utensils or visceral organs. Correspondingly, a shape of the first end of the auxiliary baffle 3 may also be circular arc to prevent causing damages to the sheet in the case that the sheet is fixed by an end or a convex surface of the auxiliary baffle 3.

Considering that the sheet (sheet-like tissues, cell membranes, etc.) are generally white, the transfer device according to the embodiment of the present disclosure may be made of a non-transparent material to facilitate the distinction between the sheet and the transfer device, so as to clearly confirm whether the sheet is on the carrying plate 11 and confirm the state of the sheet on the carrying plate 11 (e.g., whether the sheet is flattened on the carrying plate 11).

Considering that the transfer device according to the embodiment of the present disclosure is suitable for transferring the sheet, which is namely suitable for transferring the sheet-like tissues, cell membrane layers, etc. to complete various experiments, trials, clinical operations, and the like. Thus, a manufacturing material of the transfer device may include any of stainless steel, titanium alloy, primary polystyrene, high borosilicate glass and other medical materials. Of course, other materials are also available, as long as the material of the transfer device causes no damage to the sheet.

In some embodiments, the entire transfer device may be sterilized by irradiation, high temperature, or hydrogen peroxide plasma before transferring the sheet by the transfer device, so as to prevent the bacteria from contaminating the sheet and affecting the transplantation effect.

In the transfer device according to the embodiment of the present disclosure, the sheet is taken out of the transport box by the transfer device, such that the sheet is attached to the surface of the carrying plate in a flattened state during the taking out process. In addition, during the transfer process, the sheet is kept in the original shape and has no curl or tears, wrinkles, or other unrecoverable or irreversible damages, which thereby prevents the waste of resources. In addition, when it is necessary to transplant the sheet to a specific position of the surface of the visceral organ, the transfer device can transfer the sheet to the specific position of the surface of the visceral organ accurately and keep the sheet in a flattened state, which improves the transplantation efficiency.

The process of transferring or transplanting the sheet by the transfer device will be described below by taking the transfer device in FIG. 2 as an example, and the specific process is as follows.

Firstly, the sheet should be ensured in a floating sate to facilitate the picking up of the sheet. The sheet is generally in a floating state in the tissue fluid. If the sheet is not in the floating state during the transferring and is for example attached to the bottom, side wall, etc. of the transport box or vessel, the operator may shake the transport box or vessel carrying the sheet to cause the sheet to be separated from the bottom or side wall of the transport box or vessel, etc., such that the sheet turns into the floating state and the operator may pick up the sheet by the transfer device according to the embodiment of the present disclosure.

Afterwards, the operator may hold the picking-up plate 12 and move the transfer device body 1, such that the carrying plate 11 is placed into the tissue fluid and positioned below the sheet. In response to determining that the sheet is above the carrying plate 11, the operator holds the auxiliary baffle 3 and controls the auxiliary baffle 3 to abut against the third edge portion of the carrying plate 11. At this point, the transfer device body 1 and the auxiliary baffle 3 are moved at the same time until the transfer device body 1 and the auxiliary baffle 3 are separated from the tissue fluid. Then, the transfer device body 1 and the auxiliary baffle 3 are controlled to stay above the tissue fluid for a period of time until the tissue fluid on the carrying plate 11 completely flows out via the liquid leaking hole 21. At this point, the auxiliary baffle 3 may be removed or remain connected to ensure that the sheet does not slip off the carrying plate 11.

In some embodiments, the carrying plate 11 carrying the sheet is controlled to move above a specific position of the surface of the visceral organ (or above other utensils, and the specific position of the surface of the visceral organ being taken as an example to illustrate the embodiment of the present disclosure), and the carrying plate 11 is controlled to tilt at a certain angle, such that the sheet begins to slide off the carrying plate 11. In addition, the operator may also control the liquid to flow out of the liquid outlet of the buffer liquid conveying device 4, such that the sheet begins to slide off the carrying plate 11 under the driving of the liquid. In the case that a part of the sheet slides off the carrying plate 11, the operator may control an end or a convex surface of the auxiliary baffle 3 to press the part of the sheet that has been separated from the carrying plate 11 to fix the sheet at a specific position. Meanwhile, the carrying plate 11 is further controlled to tilt and move in a direction away from the specific position to allow the sheet to completely escape from the carrying plate 11 and namely to be placed on the specific position of the surface of the visceral organ completely, thereby completing the transplantation process.

In addition, although exemplary embodiments have been described herein, the scope includes any and all embodiments with equivalent elements, modifications, omissions, combinations (such as various solutions of intersected embodiments), adaptations, or changes based on the present disclosure. The elements in Claims will be interpreted broadly based on the language adopted in the claims, and are not limited to the examples described in this specification or during the implementation of the present disclosure, and the examples will be interpreted as non-exclusive. Thus, this specification and examples are intended to be regarded as examples only, and the actual scope and spirit are indicated by the overall scope of the following claims and their equivalents.

The above description is intended to be illustrative rather than restrictive. For example, the examples one or more solutions thereof) may be adopted in combination with each other. For example, those of ordinary skill in the art may adopt other embodiments while reading the above description. In addition, in the foregoing specific embodiments, various features may be grouped together to simplify the present disclosure. This should not be interpreted as an intent that an unclaimed feature as disclosed is necessary for any claim. On the contrary, the subject matter of the present disclosure may be less than all the features of a specific disclosed embodiment. Thus, the following claims are incorporated into the detailed description as examples or embodiments. Each claim herein may serve as a separate embodiment independently, and the embodiments may be combined with each other in various combinations or arrangements.

The scope of the present disclosure should be determined with reference to the appended claims and the full scope of equivalents entitled by these claims. The embodiments are only exemplary embodiments of the present disclosure, and are not indicated to limit the present disclosure. The protection scope of the present disclosure is defined by the claims. Those skilled in the art may make various modifications or equivalent substitutions to the present disclosure within the essence and protection scope of the present disclosure, and such modifications or equivalent substitutions shall also be regarded as falling within the protection scope of the present disclosure.

What is claimed is:

1. A transfer device, comprising a transfer device body, wherein
the transfer device body comprises a carrying plate and a picking-up plate; wherein
the picking-up plate is configured to drive the carrying plate to move, and a first end of the picking-up plate is fixedly connected to a first edge portion of the carrying plate; and
the carrying plate is configured to pick up and carry an object to be transplanted.

2. The transfer device according to claim 1, wherein the carrying plate and the picking-up plate are connected by a rotating mechanism, and the rotating mechanism is configured to cause, in response to being rotated, an angle between the carrying plate and the picking-up plate to change.

3. The transfer device according to claim 1, wherein the transfer device body further comprises a fixing member, wherein the fixing member is configured to fix an angle between the carrying plate and the picking-up plate to a changed angle in the case that the angle between the carrying plate and the picking-up plate is changed.

4. The transfer device according to claim 1, wherein the angle between the picking-up plate and the carrying plate is greater than 90° and less than 180°.

5. The transfer device according to claim 1, wherein the carrying plate and the picking-up plate are formed to an integral structure.

6. The transfer device according to claim 1, wherein the carrying plate is provided with at least one liquid leaking hole, wherein the liquid leaking hole is configured to allow a liquid on the carrying plate to leak.

7. The transfer device according to claim 1, wherein the transfer device body further comprises at least one blocking plate, wherein the blocking plate is configured to block the object to be transplanted from sliding out of the carrying plate, and an end of the blocking plate is fixedly connected to a second edge portion of the carrying plate.

8. The transfer device according to claim 7, wherein the first edge portion is not overlapped with the second edge portion.

9. The transfer device according to claim 1, wherein the blocking plate has at least one liquid leaking hole, wherein the liquid leaking hole is configured to allow a liquid on the carrying plate to leak.

10. The transfer device according to claim 9, wherein the liquid leaking hole is at least one of a polygonal hole and a circular hole.

11. The transfer device according to claim 8, wherein the carrying plate, the picking-up plate, and the blocking plate are formed to an integral structure.

12. The transfer device according to claim 1, further comprising: an auxiliary baffle, wherein the auxiliary baffle is configured to assist transfer of the object to be transplanted to the carrying plate.

13. The transfer device according to claim 12, wherein a shape of a first end of the auxiliary baffle is matched with a shape of a third edge portion of the carrying plate.

14. The transfer device according to claim 12, wherein the carrying plate further comprises a second edge portion configured to fix a blocking plate, and the third edge portion is not overlapped with the first edge portion and the second edge portion.

15. The transfer device according to claim 14, wherein the first edge portion, the second edge portion, and the third edge portion constitute an entire edge of the carrying plate.

16. The transfer device according to claim 1, further comprising: a buffer liquid conveying device, wherein the buffer liquid conveying device is configured to output a buffer liquid, such that the object to be transplanted moves under driving of the buffer liquid.

17. The transfer device according to claim 16, wherein the buffer liquid conveying device is fixed to the picking-up plate, and the buffer liquid delivering device is provided with a liquid outlet, wherein the liquid outlet is disposed at an end, proximal to the carrying plate, of the buffer liquid delivering device.

18. The transfer device according to claim 1, wherein the carrying plate is made of a non-transparent material.

19. The transfer device according to claim 1, wherein a carrying surface of the carrying plate is a circular surface.

20. The transfer device according to claim 1, wherein a manufacturing material of the transfer device comprises any one of stainless steel, titanium alloy, virgin polystyrene, and high borosilicate glass.
